(19) United States
(12) Patent Application Publication
(10) Pub. No.: US 2024/0287118 A1
(43) Pub. Date: Aug. 29, 2024

(54) SEPARATION PROCESS

(71) Applicant: Croda International Plc, Goole, East Yorkshire (GB)

(72) Inventor: Timothy A. Miller, Goole, East Yorkshire (GB)

(73) Assignee: Croda International Plc, Goole, East Yorkshire (GB)

(21) Appl. No.: 18/573,285

(22) PCT Filed: Jul. 11, 2022
(86) PCT No.: PCT/EP2022/069300

§ 371 Date: Dec. 21, 2023

(30) Foreign Application Priority Data
Jul. 15, 2021 (GB) 2110169.6

(51) Int. Cl. C07H 15/10; A61K 8/60; A61Q 5/02; A61Q 19/10; B01D 61/14; B01D 61/16; B01D 69/02; C07H 1/06; C11D 1/66; C12P 19/44

(52) U.S. Cl. CPC C07H 15/10; A61K 8/604; A61Q 5/02; A61Q 19/10; B01D 61/145; B01D 61/16; B01D 69/02; C07H 1/06; C11D 1/662; C12P 19/44; A61K 2800/85; B01D 2311/04; B01D 2325/02833

(57) ABSTRACT

The present invention provides the use of a separation process to obtain a product composition comprising at least one sophorolipid, wherein the product composition achieves a negative result under the h-CLAT assay according to OECD 442E, wherein the separation process comprises removing a protein component from a starting composition comprising said glycolipid to obtain said product composition, wherein said starting composition achieves a positive result under the h-CLAT assay. The invention further provides a process for obtaining a product composition comprising at least one glycolipid, a product composition obtainable from the process and a personal care formulation or home care formulation comprising the product composition.

### FIELD OF THE INVENTION

The present invention relates to the use of a separation process to produce a product composition comprising at least one glycolipid which achieves a negative result under the h-CLAT assay. The invention also relates to a process for obtaining a product composition comprising at least one glycolipid wherein the product composition achieves a negative result under the h-CLAT assay, a product composition obtainable by such a process and a personal care or home care formulation comprising such a product.

### BACKGROUND

Surfactants are amphiphilic molecules which are able to reduce interfacial and surface tension in formulations. They are commonly used within the home care and personal care industry due to their ability to remove grease and dirt from the site of application which is useful for detergents including laundry detergents, hair shampoos and soaps. Interest has grown in the use of glycolipid biosurfactants due to their improved environmental profile when compared with petrochemical derived surfactants. The group of glycolipid biosurfactants includes sophorolipids, trehalose lipids, rhamnolipids, mannosylerythritol lipids, cellobiose lipids and polyol lipids, all of which may be produced by micro-organisms.

Many consumers are becoming more aware of the ingredients used in their home care and personal care products and are selecting products based on their environmental impact. Biosurfactants are microbially produced via industrial biotechnology and/or fermentation and are able to biodegrade, which means they have an improved environmental impact when compared with petrochemical derived surfactants. Thus biosurfactants are considered as ‘green’ surfactants.

Sophorolipids are one of the most promising glycolipid biosurfactants known, one reason being their high production yield and ease of recovery from the microbial cultivation. Several Candida yeast species, including Candida bombicola (also known as Starmerella bombicola) and Candida apicola, are known to produce sophorolipids in large amounts from various substrates such as carbohydrates, vegetable oils, animal fats and n-alkanes.

There is a commercial need for the development of improved glycolipid compositions.

### SUMMARY OF THE INVENTION

It is an object of the present invention to address the above and/or other disadvantages associated with the prior art.

According to a first aspect, the present invention provides the use of a separation process to obtain a product composition comprising at least one glycolipid which achieves a negative result under the h-CLAT assay according to OECD 442E, wherein the separation process comprises removing a protein component from a starting composition comprising said glycolipid to obtain said product composition, wherein said starting composition achieves a positive result under the h-CLAT assay.

According to a second aspect, the present invention provides a process for obtaining a product composition comprising at least one glycolipid, comprising the steps of:
a) obtaining a starting composition from a fermentation process wherein i) the starting composition is a crude composition obtainable directly from the fermentation process, or ii) the starting composition is obtainable from the crude composition after at least one further processing step selected from water washing, hydrolysis, centrifugation and precipitation; wherein the starting composition comprises at least one glycolipid and a protein component; and
b) separating the protein component from the starting composition by a step of ultra-filtration using a membrane with pore size from 0.01 to 0.1 μm to obtain a product composition comprising at least one glycolipid;
wherein the presence of the protein component causes the starting composition to achieve a positive result under the h-CLAT assay according to OECD 442E; and wherein the product composition achieves a negative result under the h-CLAT assay.

According to a third aspect, the present invention provides a product composition obtainable by a process according to the second aspect, wherein the product composition comprises at least one glycolipid and wherein the product composition achieves a negative result under the h-CLAT assay.

According to a fourth aspect, the present invention provides a personal care formulation comprising a product composition according to the third aspect.

According to a fifth aspect, the present invention provides a home care formulation comprising a product composition according to the third aspect.

The present invention is based in part on the recognition by the inventors that a disadvantage of prior art glycolipid compositions is that they can result in a positive result under the h-CLAT assay according to OECD 442E, which may be an indication of a potential skin sensitising effect when considered in combination with the Direct Peptide Reactivity Assay (DPRA) according to OECD 442C and the LuSens or Luciferase Keratinocyte Activation assay according to OECD 442D. The inventors have surprising found that removal of a protein component from a glycolipid containing composition allows the resulting product composition to achieve a negative h-CLAT assay. The product composition (without the protein component) is therefore improved by having a negative h-CLAT assay and therefore having an improved (lower) risk of skin sensitisation. This lower skin sensitisation risk is desirable in many application areas, for example consumer applications including in personal care formulations and in home care formulations.

A further advantage of the invention is that the colour of the product composition may be improved (made lighter) compared with the starting composition. For example, the product composition may have a lower (lighter) value on the Gardner colour scale (Gardner colour).

All of the features described herein may be present in any of the above aspects of the invention, in any combination.

### BRIEF DESCRIPTION OF THE DRAWINGS

FIG. 1 shows a flowchart of the process steps taken to obtain comparative Samples A to D as described in Example 1 and the steps for Sample 1 according to the invention as described in Example 2.

### DETAILED DESCRIPTION OF THE INVENTION

It will be understood that any upper or lower quantity or range limit used herein may be independently combined.

It will be understood that, when describing the number of carbon atoms in a substituent group (e.g. ‘C1 to C6’), the number refers to the total number of carbon atoms present in the substituent group, including any present in any branched groups. Additionally, when describing the number of carbon atoms in, for example fatty acids, this refers to the total number of carbon atoms including the one at the carboxylic acid, and any present in any branch groups.

Many of the chemicals which may be used to produce the compounds and compositions of the present invention are obtained from natural sources. Such chemicals typically include a mixture of chemical species due to their natural origin. Due to the presence of such mixtures, various parameters defined herein can be an average value and may be non-integral.

It is understood that the terms Candida bombicola and Starmerella bombicola refer to the same micro-organism species but have been used interchangeably at different times in different references.

It is understood that the term ‘crude composition’ refers to a composition comprising at least one glycolipid which is obtained directly from a fermentation process. A decanting or decantation step may be used to obtain the crude composition from the fermentation process.

It is understood that the term ‘starting composition’ refers to a composition comprising at least one glycolipid which may be a crude composition or may have been further processed from the crude composition but has not been through the separation process of the invention.

It is understood that the term ‘product composition’ refers to a starting composition which has been through the separation process of the invention.

The term ‘glycolipid’ when used herein means a compound comprising one or more monosaccharide residues bound by a glycosidic linkage to a hydrophobic moiety such as a lipid or hydrocarbyl group. Glycolipids include sophorolipids, trehalose lipids, rhamnolipids, mannosylerythritol lipids, cellobiose lipids and polyol lipids. Preferably the glycolipid is selected from a sophorolipid, trehalose lipid, rhamnolipid or mannosylerythritol lipid, more preferably selected from a sophorolipid, rhamnolipid or mannosylerythritol lipid, particularly preferably selected from a sophorolipid or rhamnolipid.

The term ‘solids content’ means the percentage by weight of material in a composition which is solids i.e. is non-volatile and which remains in a sample of the composition after oven drying to remove all water and any other volatile components present in the composition.

The term ‘personal care formulation’ means a product intended to be applied to the human body or any part thereof for cleansing, beautifying or improving appearance. Personal care formulations include but are not limited to hand soaps; bar soaps; liquid soaps; facial and body washes; facial and body cleansers; shampoos; conditioners; toothpaste; shaving creams or gels; and foot care products. A personal care formulation does not include any product for which a prescription is required.

The term ‘home care formulation’ means a product use by household and institutional consumers for cleaning, caring or conditioning of the home or any of its contents including, but not limited to, detergents including laundry detergents and dishwashing detergents; cleaning compounds including hard surface cleaners; polishes and floor finishes.

### Glycolipid

Preferably the at least one glycolipid of the invention is a biosurfactant.

Preferably the at least one glycolipid is selected from the group of sophorolipids, trehalose lipids, rhamnolipids, mannosylerythritol lipids, cellobiose lipids and polyol lipids. Preferably the glycolipid is selected from sophorolipids, trehalose lipids, rhamnolipids and mannosylerythritol lipids, more preferably selected from sophorolipids, rhamnolipids and mannosylerythritol lipids, particularly preferably selected from sophorolipids and rhamnolipids. Preferably the at least one glycolipid is at least one sophorolipid.

The producing organism which makes the glycolipid may be selected from bacteria, fungi and yeasts. The glycolipid may be produced by a fermentation process. The fermentation process may use a fermentation mixture comprising the producing organism and a substrate.

Sophorolipid Preferably the at least one glycolipid comprises at least one sophorolipid. The at least one sophorolipid may be produced by any of the producing organisms described in Table 1 which are listed in order of yield from highest to lowest.

TABLE 1
Sophorolipid producing organisms

| Producing Organism | Typical Yield (g/L) |
|---|---|
| Starmerella bombicola | 300 |
| Candida albicans | 108.7 |
| Wickerhamiella | 53.7 |
| domericqiae | |
| Candida apicola | 52.7 |
| Candida floricola | 22.9 |
| Candida kuoi | 20.1 |
| Candida stellata | 11.9 |
| Candida riodocensis | 8.9 |
| Candida batistae | 6 |
| Candida tropicalis | ND |
| Cyberlindera | ND |
| samutprakarnensis | |

*ND = not determined

The sophorolipid may be obtainable, preferably is obtained by a fermentation process. The sophorolipid may be obtainable from a natural or modified micro-organism, preferably selected from the group of Starmerella (or Candida) bombicola, Candida albicans, Wickerhamiella domericqiae, Candida apicola, Candida floricola, Candida kuoi, Candida stellata, Candida riodocensis, Candida batistae, Candida tropicalis, Cyberlindera samutprakarnensis. Preferably the sophorolipid is obtainable from Starmerella bombicola.

In a sophorolipid obtainable from Starmerella bombicola, the hydrophilic moiety of the biosurfactant molecule is a disaccharide (i.e., sophorose), and the hydrophobic portion is an omega- or (omega-1)-hydroxy fatty acid attached to the sophorose via a glycosidic bond. The fatty acid chain, most commonly containing 16- and 18-carbon atoms, may be unsaturated and lactonized to the disaccharide. There are generally considered to be two types of sophorolipids, the acid form (open-chain) and the lactone form (closed-chain) sophorolipids. To produce a lactone form sophorolipid, the hydroxyl fatty acid moiety of the acid form sophorolipids forms a macrocyclic lactone ring with the 4″-hydroxyl group of the sophorose by intramolecular esterification.

### Crude Composition

The crude composition is a composition comprising at least one glycolipid, preferably at least one sophorolipid, which is obtained directly from a fermentation process. A decanting or decantation step may be used to obtain the crude composition from the fermentation process.

The fermentation process from which the crude composition is obtainable typically utilises sugars and alkanes or lipids as substrates. The fermentation process may use a fermentation mixture comprising the producing organism and a substrate. Appropriate fermentation processes are reviewed in A P Tulloch, J F T Spencer and P A J Gorin, Can. J Chem (1962) 40 1326 and U Gobbert, S Lang and F Wagner, Biotechnology Letters (1984) 6 (4), 225.

Preferably the fermentation process uses Starmerella bombicola as the producing organism. Preferably the fermentation process uses glucose and/or oleic acid as substrate. The oleic acid may be an enriched fraction of vegetable origin. The fermentation process may use ammonium sulfate. The fermentation process may use antifoam. The fermentation process may be carried out at a temperature of 25 to 30° C., preferably about 27° C. The fermentation process may be carried out at a pH of 3 to 6, preferably 3.5 to 5.7. The fermentation process may be carried out at a pressure of 0.4 to 0.8 bar, preferably about 0.5 bar. The fermentation process may be carried out at a dissolved oxygen of at least 20%, preferably at least 25%, more preferably at least 30%. The dissolved oxygen level may be maintained by constant stirrer speed and air flow rate. The fermentation process preferably does not comprise additional oxygen enrichment.

### Starting Composition

The starting composition of the invention comprises at least one glycolipid, preferably at least one sophorolipid.

The starting composition may be obtainable from a fermentation process, preferably a fermentation process involving Starmerella bombicola, wherein i) the starting composition is a crude composition obtainable directly from the fermentation process, or ii) the starting composition is obtainable from the crude composition after at least one further processing step selected from water washing, hydrolysis, centrifugation, precipitation. The hydrolysis may be partial or full hydrolysis, preferably the hydrolysis is alkali hydrolysis. Preferably the precipitation is achieved by pH adjustment to adjust the solubility of the glycolipid.

The starting composition may be obtainable by a process comprising fermentation and at least one further step selected from washing with water, solubilising by pH adjustment, centrifuging, hydrolysis. Preferably the starting composition is obtainable by a process comprising fermentation and a further step of hydrolysis, preferably partial hydrolysis preferably partial alkali hydrolysis.

Preferably the starting composition has a Gardner colour of at least 6.5, more preferably at least 7, yet more preferably at least 7.5. The starting composition may have a Gardner colour of at most 18, preferably at most 15, more preferably at most 12. Preferably the Gardner colour is measured as described herein.

Preferably the starting composition comprises at least 10 wt % glycolipid, preferably at least 20 wt %, more preferably at least 30 wt %, particularly preferably at least 40 wt % wherein the wt % is on the basis of the total weight of the composition. Preferably the starting composition comprises at most 80 wt % glycolipid, preferably at most 70 wt %, more preferably at most 60 wt %, desirably at most 55 wt % wherein the wt % is on the basis of the total weight of the composition.

Preferably the at least one glycolipid in the starting composition comprises a mixture of acid form (open-chain) sophorolipids and lactone form (closed-chain) sophorolipids. The sophorolipid component of the starting composition preferably comprises at least 40 wt %, more preferably at least 50 wt %, particularly preferably at least 60 wt %, desirably at least 65 wt % of acid form sophorolipid on the basis of the total weight of sophorolipid in the composition. The sophorolipid component of the starting composition preferably comprises at most 60 wt %, more preferably at most 50 wt %, particularly preferably at most 40 wt %, desirably at most 35 wt % of lactone form sophorolipid on the basis of the total weight of sophorolipid in the composition.

Preferably the starting composition comprises water. Preferably the starting composition comprises at least 10 wt % water, preferably at least 20 wt %, more preferably at least 30 wt %, particularly preferably at least 40 wt % wherein the wt % is on the basis of the total weight of the composition. Preferably the starting composition comprises at most 80 wt % water, preferably at most 70 wt %, more preferably at most 60 wt %, wherein the wt % is on the basis of the total weight of the composition.

The starting composition comprises a protein component which, without being bound by theory, is believed to cause the starting composition to achieve a positive result under the h-CLAT assay as described herein. The protein component may comprise an enzyme. Preferably the protein component has a molecular weight in the range 20 kDa to 200 kDa, measured by SDS-PAGE as described herein. Preferably the protein component has a molecular weight of at least 25 kDa, more preferably at least 30 kDa, yet more preferably at least 40 kDa, particularly at least 45 kDa measured by SDS-PAGE as described herein. Preferably the protein component has a molecular weight of at most 190 kDa, more preferably at most 180 kDa, yet more preferably at most 170 kDa, particularly at most 160 kDa measured by SDS-PAGE as described herein.

Preferably the starting composition has a protein concentration of at least 10 mg of protein per g of solids (mg/g), preferably at least 15 mg/g, more preferably at least 20 mg/g on the basis of the total weight of protein and the total weight of solids in the product composition.

Preferably the total weight of protein is measured by BCA assay as described herein. Preferably the total weight of solids is measured as described herein.

### Separation Process

The separation process of the invention preferably comprises a step of micro-filtration. The micro-filtration may comprise a membrane with a pore size from 0.1 to 10 μm, preferably from 0.15 to 5 μm, more preferably from 0.15 to 1 μm, particularly preferably from 0.15 to 0.5 μm. The micro-filtration may remove large debris from the starting composition. The micro-filtration membrane may be a polyethylsulfone membrane or a ceramic membrane, preferably a ceramic membrane.

The separation process of the invention preferably comprises a step of ultra-filtration. The ultra-filtration may use a membrane with a pore size from 0.01 to 0.1 μm, preferably from 0.03 to 0.05 μm. The ultra-filtration membrane preferably has a pore size of at least 0.01 μm, more preferably at least 0.02 μm, yet more preferably at least 0.03 μm. The ultra-filtration membrane preferably has a pore size of at most 0.09 μm, more preferably at most 0.08 μm, yet more preferably at most 0.07 μm, particularly at most 0.06 μm, especially at most 0.05 μm. Preferably the ultra-filtration step removes the protein component. The ultra-filtration membrane material may be selected from cellulose, polyethylsulfone and polysulfone.

The separation process of the invention preferably comprises a step of nano-filtration. The nano-filtration may comprise a membrane with a pore size from 0.001 to 0.01 μm, preferably from 0.001 to 0.009 μm. The nano-filtration membrane preferably has a pore size of at least 0.001 μm, more preferably at least 0.002 μm, yet more preferably at least 0.003 μm. The nano-filtration membrane preferably has a pore size of at most 0.007 μm, more preferably at most 0.005 μm, yet more preferably at most 0.003 μm. The nano-filtration membrane may remove water and/or salt from the composition. Preferably the nano-filtration membrane concentrates the composition.

Preferably the separation process comprises a step of ultra-filtration and a step of nano-filtration. Preferably the separation process comprises a step of micro-filtration, a step of ultra-filtration and a step of nano-filtration.

Preferably the separation process does not comprise a step of washing with a hydrocarbon solvent, more preferably the separation process does not comprise a step of washing with hexane.

### Product Composition

The product composition of the invention comprises at least one glycolipid and achieves a negative result under the h-CLAT assay as described herein. Preferably the at least one glycolipid comprises or is at least one sophorolipid.

Preferably the product composition is obtainable by a process as described herein wherein the product composition comprises at least one glycolipid and wherein the product composition achieves a negative result under the h-CLAT assay.

Preferably the product composition comprises a protein concentration of at most 5 mg of protein per g of solids (mg/g), preferably at most 3 mg/g, more preferably at most 1 mg/g on the basis of the total weight of protein and the total weight of solids in the product composition.

Preferably the product composition has a Gardner colour of at most 6, more preferably at most 5, yet more preferably at most 4. Preferably the product composition has a Gardner colour of at least 1, more preferably at least 2.

A disadvantage of some prior art glycolipid compositions is that they achieve a positive result under the h-CLAT assay, for example as shown in Table 5. Without being bound by theory, a positive h-CLAT may indicate a higher risk of a skin sensitising effect in those prior art compositions. The product composition of the invention has a protein component removed from it which is believed to be the cause of the positive h-CLAT. Therefore the product composition (without the protein component) is advantageous in achieving a negative h-CLAT and therefore having an improved (lower) risk of skin sensitisation. This lower skin sensitisation risk is desirable in many application areas, for example consumer applications including personal care formulations and home care formulations.

Preferably the product composition comprises at least 10 wt % glycolipid, preferably at least 20 wt %, more preferably at least 30 wt %, particularly preferably at least 40 wt % wherein the wt % is on the basis of the total weight of the composition. Preferably the product composition comprises at most 70 wt % glycolipid, preferably at most 60 wt %, more preferably at most 55 wt %, particularly preferably at most 50 wt % wherein the wt % is on the basis of the total weight of the composition.

Preferably the at least one glycolipid in the product composition comprises a mixture of acid form (open-chain) sophorolipids and lactone form (closed-chain) sophorolipids. The sophorolipid component of the starting composition preferably comprises at least 40 wt %, more preferably at least 50 wt %, particularly preferably at least 60 wt %, desirably at least 65 wt % of acid form sophorolipid on the basis of the total weight of sophorolipid in the composition. The sophorolipid component of the starting composition preferably comprises at most 60 wt %, more preferably at most 50 wt %, particularly preferably at most 40 wt %, desirably at most 35 wt % of lactone form sophorolipid on the basis of the total weight of sophorolipid in the composition.

The product composition may comprise water, preferably the product composition comprises at most 90 wt % water, more preferably at most 80 wt % water, yet more preferably at most 70 wt % water, particularly preferably at most 60 wt % water on the basis of the total weight of the product composition. Preferably the product composition comprises at least 30 wt % water, more preferably at least 40 wt % water, yet more preferably at least 45 wt % water, particularly preferably at least 50 wt % water on the basis of the total weight of the product composition.

Preferably the product composition consists essentially of glycolipid, preferably sophorolipid, and water. An advantage of the product composition over known glycolipid compositions may be a lower amount of other components in the composition apart from glycolipid and water as a result of the separation process.

The product composition may comprise at most 5 wt % of unconverted substrate from the fermentation process, preferably at most 1 wt %, more preferably at most 0.2 wt %, on the basis of the total weight of the product composition.

The product composition may comprise at most 5 wt % of fatty acid, preferably oleic acid, preferably at most 1 wt %, more preferably at most 0.2 wt %, on the basis of the total weight of the product composition. The product composition may comprises substantially no fatty acid, preferably comprises no fatty acid.

Preferably the product composition is obtainable by a process as described herein.

### Use of the Invention

According to one aspect, the invention provides the use of a separation process to obtain a product composition comprising at least one sophorolipid, wherein the product composition achieves a negative result under the h-CLAT assay according to OECD 442E, wherein the separation process comprises removing a protein component from a starting composition comprising said sophorolipid to obtain said product composition, wherein said starting composition achieves a positive result under the h-CLAT assay.

Preferably the protein component has a molecular weight in the range 20 kDa to 200 kDa as measured by SDS-PAGE.

Preferably the product composition has a protein concentration of at most 5 mg of protein per g of solids (mg/g), preferably at most 3 mg/g, more preferably at most 1 mg/g, particularly at most 0.5 mg/g on the basis of the total weight of protein and the total weight of solids in the product composition.

Preferably the product composition has a Gardner colour of at most 5.

Preferably the starting composition is obtainable from a fermentation process, preferably a fermentation process involving Starmerella bombicola, wherein i) the starting composition is a crude composition obtainable directly from the fermentation process, or ii) the starting composition is obtainable from the crude composition after at least one further processing step selected from water washing, hydrolysis, centrifugation and precipitation.

Preferably the separation process comprises a step of ultra-filtration using a membrane with pore size from 0.01 to 0.1 μm.

Preferably the separation process does not comprise a step of washing with a hydrocarbon solvent.

The use may comprise any of the other aspects or features of the invention described herein.

### Process of the Invention

According to one aspect, the invention provides a process for obtaining a product composition comprising at least one glycolipid, comprising the steps of:
a) obtaining a starting composition from a fermentation process wherein i) the starting composition is a crude composition obtainable directly from the fermentation process, or ii) the starting composition is obtainable from the crude composition after at least one further processing step selected from water washing, hydrolysis, centrifugation and precipitation; wherein the starting composition comprises at least one glycolipid and a protein component; and
b) separating the protein component from the starting composition by a step of ultra-filtration using a membrane with pore size from 0.01 to 0.1 μm to obtain a product composition comprising at least one glycolipid;
wherein the presence of the protein component causes the starting composition to achieve a positive result under the h-CLAT assay according to OECD 442E; and wherein the product composition achieves a negative result under the h-CLAT assay.

The process may comprise any of the other aspects or features of the invention described herein.

### Personal Care & Home Care Formulations

According to one aspect, the invention provides a personal care formulation comprising a product composition of the invention.

The personal care formulation may be selected from hand soaps; bar soaps; liquid soaps; facial and body washes; facial and body cleansers; shampoos; conditioners; toothpaste; shaving creams or gels; foot care products, moisturizers, sunscreens, after sun products, body butters, gel creams, high perfume containing products, perfume creams, baby care products, hair treatments, hair colourants, skin toning and skin whitening products, water-free products, anti-perspirant and deodorant products, tanning products, cleansers, 2-in-1 foaming emulsions, multiple emulsions, preservative free products, mild formulations, scrub formulations e.g. containing solid beads, silicone in water formulations, pigment containing products, sprayable emulsions, cosmetics, colour cosmetics, conditioners, shower products, foaming emulsions, make-up remover, eye make-up remover, and wipes.

The personal care formulation may be a spray, lotion, cream or ointment. When the formulation is a colour cosmetic, it may be a foundation, mascara, eyeshadow or lipstick. The personal care formulation may be an anti-perspirant or deodorant.

The personal care formulation may have a pH value over a wide range, preferably in the range from 3 to 13, more preferably 4 to 10, and especially 5 to 8.

According to one aspect, the invention provides a home care formulation comprising a product composition of the invention.

The home care formulation may be selected from fabric cleaners, fabric conditioners, stain removers, laundry detergents, hard surface cleaners, dishwashing detergents, machine dishwashing detergents, polishes and floor finishes.

In addition to the glycolipid, the home care formulation and/or personal care formulation may comprise a further surfactant. The further surfactant may be selected from non-ionic surfactants, anionic surfactants, cationic surfactants and mixtures thereof.

Any of the features described herein may be taken in any combination and with any aspect of the invention.

### EXAMPLES

The invention is illustrated by the following non-limiting examples. All parts and percentages are given by weight unless otherwise stated.

It will be understood that all tests and physical properties listed have been determined at atmospheric pressure and ambient/room temperature (i.e. about 23-25° C.), unless otherwise stated herein, or unless otherwise stated in the referenced test methods and procedures.

### Test Methods

In this specification the following test methods have been used:

### (i) SDS-PAGE

SDS-PAGE (sodium dodecyl sulphate-polyacrylamide gel electrophoresis) gels were performed according to the following method to detect the presence of protein in a sample. The sample was washed with ethyl acetate to separate glycolipids from protein. The ethyl acetate layer is then removed, and the water layer precipitated by the TCA method. The pellet is subsequently washed three times with chilled acetone to remove any remaining traces of glycolipid. The sample was run on a 4-20% triceine TruPAGE gels according to the standard procedure (TruPAGE Precast Gel System—Sigma Aldrich). The gels were stained with EZBlue Gel Staining Reagent. Visual inspection of the gel was used to determine whether any protein was present.

### (ii) BCA Protein Assay

Bicinchoninic acid (BCA) assay or Smith assay is a copper-based colorimetric assay for the quantification of the total weight of protein in a sample. The BCA assay relies on the formation of a Cu²⁺-protein complex in a basic environment, followed by reduction of the Cu²⁺ to Cu⁺ (Smith et al., 1985). The amount of Cu²⁺ that is reduced is proportional to the amount of protein present in solution.

(iv) h-CLAT

The human Cell Line Activation Test (h-CLAT) is a cell-based assay that identifies skin sensitizers by examining changes in the expression of cell surface markers (CD54 and CD86) implicated in dendritic cell activation, the third key event of the skin sensitization AOP. Following exposure of the THP-1 human monocyte cell line to the test substance, expression levels of CD54 and CD86 are quantified by flow cytometry and compared to controls. This assay follows the accepted OECD guideline (OECD 442E) which defines the conditions for a negative or positive result under this assay.

### (v) Solids Content

Solids content was measured for a known weight of sample by oven drying at 105° C. for 17 19 hours to remove the moisture and any other volatile components present. After cooling in a desiccator, the residual weight is used to calculate the solids content (in wt %) of the sample.

### (vi) Gardner Colour

The Gardner colour of a sample was measured automatically using a Lovibond PFXi Spectrocolourimeter and 10 mm pathlength glass sample cell.

### Example 1

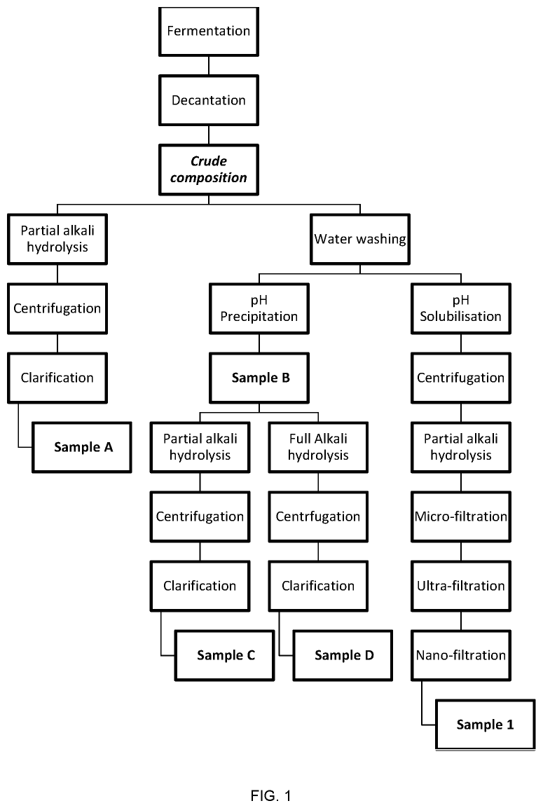

Various comparative samples were produced from a fermentation process via a crude composition comprising a sophorolipid which was then further treated to produce the Samples A to D. FIG. 1 shows the fermentation and decantation steps which produce the crude composition and the further steps to produce comparative Samples A to D.

### Crude Composition

The crude sophorolipid composition was produced as follows. A standard fermentation process using Starmerella bombicola with glucose and oleic acid is used. The fermentation process is conducted in a yeast extract (1.5% w/v)/ammonium sulfate (0.28% w/v)/antifoam (0.625% v/v) medium at a temperature of 27° C., pH 5.7-3.5, 0.5 bar and dissolved oxygen >30%. The oxygen level is maintained by constant stirrer speed and air flow rate without further oxygen enrichment. The process is fed-batch with continuous glucose and oleic acid feeds. Upon completion, the fermentation process produced a composition with two separate phases. One of the phases contained the produced sophorolipid and will be referred to as the crude sophorolipid composition. The crude sophorolipid composition had a solids content of at least 45 wt % and a remainder consisting essentially of water. The separated crude sophorolipid composition was decanted from the fermenter as shown in FIG. 1.

The sophorolipid component of the crude sophorolipid composition is typically at least 70 wt % lactone form sophorolipid, on the basis of the total weight of sophorolipid with the remaining sophorolipid being acid form.

The trace oleic acid concentration in the crude sophorolipid composition was less than 1 wt %.

### Comparative Starting Compositions

FIG. 1 shows the process steps used to make the following comparative starting compositions from the crude sophorolipid composition:
Sample A This partially hydrolysed sophorolipid was produced by partial alkali hydrolysis of the crude composition to provide a mixture of acid form sophorolipid and lactone form sophorolipid at a weight ratio of approximately 70:30. The mixture was then centrifuged and clarified. The resulting Sample A had a dark amber colour with a Gardner colour value of at least 7.
Sample B This lactone form sophorolipid was produced by water washing the crude sophorolipid composition and adjusting the pH to 5.5 to precipitate the lactone form sophorolipid and produce a white slurry containing pure lactone form sophorolipid which was separated and dried to produce Sample B.
Sample C This sophorolipid was produced by partial alkali hydrolysis of Sample B to provide an acid form to lactone form weight ratio of approximately 70:30. The mixture was then centrifuged and clarified to produce Sample C.
Sample D This fully hydrolysed sophorolipid was produced by full alkali hydrolysis of Sample B to provide an acid form to lactone form weight ratio of approximately 100:0. The mixture was then centrifuged and clarified to produce Sample D.

As can be seen in Table 5, Samples A to D all achieve a positive result under the h-CLAT assay described in the Test Methods. Without being bound by theory, a positive result under the h-CLAT assay may indicate a higher potential risk of a skin sensitisation effect than a negative result.

### Example 2

A product composition according to the invention, Sample 1, was made as follows.

The same crude sophorolipid composition from Example 1 was used. This was then washed with water, solubilised by pH adjustment to 7.5, centrifuged at 14000 rpm for 30 minutes and partially alkali hydrolysed to give a starting composition with an acid form sophorolipid to lactone form weight ratio of approximately 70:30. This starting composition had a solids content of 20 wt % approx.

### Separation Process

The starting composition was then treated with a micro-filtration step using a 0.22 μm steri-cup. This was followed by an ultra-filtration step using a membrane with a pore size of 0.05 μm and then a nano-filtration step using a membrane with a pore size of 0.001 μm. This yielded a product composition, Sample 1, which had a solids content of 15 wt %.

### Example 3

The comparative Samples A to D of Example 1 and Sample 1 according to the invention were tested at various stages of their production process for the presence of a protein component via visual inspection of SDS-PAGE gels as described in the Test Methods. The presence of a protein component on the SDS-PAGE gel is indicated as (+) in Tables 2 to 4. The samples were also tested at various stages for protein concentration via the BCA protein assay and tested with the h-CLAT assay as described in the Test Methods. The results are given in Tables 2 to 4.

TABLE 2
Production of Sample A

| | Presence of | Protein | |
|---|---|---|---|
| | protein via | concentration | h-CLAT |
| Composition or | SDS-PAGE | via BCA assay | assay |
| Processing Step | (+/−) | (mg/g solids) | (+/−) |
| Crude | + | 20.03 | ND |
| Composition | | | |
| Partial alkali | + | ND | ND |
| hydrolysis | | | |
| Centrifugation | + | ND | ND |
| Clarification | + | ND | ND |
| Sample A | + | ND | + |

*ND = not determined

TABLE 3
Production of Samples B to D

| | Presence of | | |
|---|---|---|---|
| | protein | Protein | |
| | component | concentration | h-CLAT |
| Composition or | via SDS- | via BCA assay | assay |
| Processing Step | PAGE (+/−) | (mg/g solids) | (+/−) |
| Crude | + | 20.03 | ND |
| Composition | | | |
| Precipitation at pH | ND | ND | ND |
| 5.5 | | | |
| Sample B | ND | ND | + |
| Sample C | ND | ND | + |
| Sample D | ND | ND | + |

*ND = not determined

TABLE 4
Production of Sample 1 of Example 2

| | Presence of | | |
|---|---|---|---|
| | protein | Protein | |
| | component | concentration | h-CLAT |
| Composition or | via SDS- | via BCA assay | assay |
| Processing Step | PAGE (+/−) | (mg/g solids) | (+/−) |
| Crude | + | 20.03 | ND |
| composition | | | |
| Water washing | + | 2.4 | ND |
| pH Solubilisation | + | ND | + |
| Centrifugation | + | ND | ND |
| Partially alkali | + | 1.61 | ND |
| hydrolysis | | | |
| Micro-filtration | + | ND | ND |
| Ultra-Filtration | − | 0.14 | ND |
| Nano-filtration | − | ND | ND |
| Sample 1 | − | ND | − |

*ND = not determined

It can be seen from Tables 2 to 4 that Sample 1 from Example 2 is the only sample which provides a negative result under the h-CLAT assay. This is summarised in Table 5.

TABLE 5
h-CLAT assay results from Tables 2 to 4

| Sample | h-CLAT |
|---|---|
| Sample A | + |
| Sample B | + |
| Sample C | + |
| Sample D | + |
| Sample 1 | − |

It can be seen from Table 5 that all the sample compositions from Example 1 achieved a positive result under the h-CLAT assay which may indicate a higher risk of a potential skin sensitising effect. In contrast, Sample 1 of the invention following the separation process of Example 2 achieved a negative result under the h-CLAT assay. Without being bound by theory, a negative result under h-CLAT should indicate an improved (lower) risk of skin sensitisation. Thus the composition of Example 2, after the separation process of the invention, may advantageously be used in applications where skin sensitisation is of concern such as home care formulations and personal care formulations.

### Example 4

The following personal care and home care formulations are provided as examples of formulations which include Sample 1 of Example 2 according to the invention.

TABLE 6
Facial Cleanser

| Ingredient (INCI) | Function | w/w % |
|---|---|---|
| Water Deionised (Aqua) | Solvent | To 100 |
| Pricerine ™ 9091 (Glycerin) | Humectant | 2 |
| Crodasinic LS30 (Water (and) Sodium | Anionic | 10 |
| Lauroyl Sarcosinate) | surfactant | |
| Sample 1 | Surfactant | 7.5 |
| Incromine Oxide C (Cocamidopropylamine | Non-ionic | 5 |
| Oxide (and) Water (aqua)) | surfactant | |
| Phytofoam (Water (and) Acacia Concinna | Foaming agent | 4 |
| Fruit Extract (and) Balanites Aegyptiaca | | |
| (Desert Date) Fruit Extract (and) Gypsophila | | |
| Paniculata Root Extract) | | |
| Phytessence Speedwell (Water (and) | Cleansing | 1 |
| Glycerin (and) Veronica Officinalis | infusion extract | |
| Flower/Leaf/Stem Extract) | | |
| Natragem S140 (Polyglyceryl-4 | Solubiliser | 2 |
| Laurate/Sebacate (and) Polyglyceryl-6 | | |
| Caprylate/Caprate (and) Aqua) | | |
| Cromollient SCE (Di-PPG-2 Myreth-10 | Emollient | 1 |
| Adipate) | | |
| Euxyl PE9010 (Phenoxyethanol (and) | Preservative | 0.5 |
| Ethylhexylglycerin) | | |

TABLE 7
Shampoo

| Ingredient (INCI) | Function | w/w % |
|---|---|---|
| Water Deionised (Aqua) | Solvent | To 100 |
| Plantaren 200N (Alkylpolyglucoside) | Non-ionic | 15 |
| | surfactant | |
| Plantacare 818 UP (Alkylpolyglucoside) | Non-ionic | 10 |
| | surfactant | |
| Sample 1 | Surfactant | 2.5 |
| Arlasilk EFA (Linoleamidopropyl PG- | Conditioning | 2 |
| Dimonium Chloride Phosphate) | agent | |
| Arlasilk PTM (Myristamidopropyl PG- | Conditioning | 2 |
| Dimonium Chloride Phosphate) | agent | |
| Promidium LTS (PEG-150 Distearate (and) | Rheology | 3.5 |
| PPG-2 Hydroxyethyl Cocamide) | modifier | |
| Euxyl PE9010 (Phenoxyethanol (and) | Preservative | 0.5 |
| Ethylhexylglycerin) | | |

TABLE 8
Emulsion

| Ingredient (INCI) | Function | w/w % |
|---|---|---|
| Water Deionised (Aqua) | Solvent | To 100 |
| Pricerine ™ 9091 (Glycerin) | Humectant | 3 |
| Sample 1 | O/W Emulsifier | 5 |
| Arlamol HD (Isohexadecane) | Emollient | 5 |
| Crodamol IPM (Isopropyl Myristate) | Emollient | 4 |
| Arlamol PS15E (PPG-15 Stearyl Ether) | Emollient | 3 |
| Crodacol ™CS90 (Cetearyl Alcohol) | Viscosity builder | 2 |
| Keltrol CG-T (Xanthan Gum) | Rheology Modifier | 0.8 |
| Euxyl PE9010 (Phenoxyethanol (and) | Preservative | 0.5 |
| Ethylhexylglycerin) | | |

TABLE 9
All-Purpose Home Care Cleaner

| Ingredient | w/w % |
|---|---|
| Water Deionised (Aqua) | To 100 |
| NatraSense AG-810 (ex Croda) | 1.5 |
| Sample 1 | 2.5 |
| Sodium citrate | 1.0 |
| Sodium gluconate | 0.5 |

TABLE 10
Bathroom Cleaner

| Ingredient | w/w % |
|---|---|
| Water Deionised (Aqua) | To 100 |
| NatraSense AG-810 (ex Croda) | 1.5 |
| Sample 1 | 2.5 |
| Citric acid | 1.0 |
| Sodium citrate | to pH 9 |

It is to be understood that the invention is not to be limited to the details of the above embodiments, which are described by way of example only. Many variations are possible.

1. Use of a separation process to obtain a product composition comprising at least one sophorolipid, wherein the product composition achieves a negative result under the h-CLAT assay according to OECD 442E, wherein the separation process comprises removing a protein component from a starting composition comprising said sophorolipid to obtain said product composition, wherein said starting composition achieves a positive result under the h-CLAT assay, wherein the separation process does not comprise a step of washing with a hydrocarbon solvent.

2. Use according to claim 1 wherein the protein component has a molecular weight in the range 20 kDa to 200 kDa as measured by SDS-PAGE.

3. Use according to claim 1 wherein the product composition has a protein concentration of at most 5 mg of protein per g of solids (mg/g) on the basis of the total weight of protein and the total weight of solids in the product composition.

4. Use according to claim 1 wherein the product composition has a Gardner colour of at most 5.

5. Use according to claim 1 wherein the starting composition is obtainable from a fermentation process, preferably a fermentation process involving Starmerella bombicola, wherein i) the starting composition is a crude composition obtainable directly from the fermentation process, or ii) the starting composition is obtainable from the crude composition after at least one further processing step selected from water washing, hydrolysis, centrifugation and precipitation.

6. Use according to claim 1 wherein the separation process comprises a step of ultra-filtration using a membrane with pore size from 0.01 to 0.1 μm.

7. A process for obtaining a product composition comprising at least one glycolipid, comprising the steps of:
a) obtaining a starting composition from a fermentation process wherein i) the starting composition is a crude composition obtainable directly from the fermentation process, or ii) the starting composition is obtainable from the crude composition after at least one further processing step selected from water washing, hydrolysis, centrifugation and precipitation;
wherein the starting composition comprises at least one glycolipid and a protein component; and
b) separating the protein component from the starting composition by a step of ultra-filtration using a membrane with pore size from 0.01 to 0.1 μm to obtain a product composition comprising at least one glycolipid;
wherein the presence of the protein component causes the starting composition to achieve a positive result under the h-CLAT assay according to OECD 442E; and
wherein the product composition achieves a negative result under the h-CLAT assay.

8. A process according to claim 7 wherein the at least one glycolipid comprises at least one sophorolipid.

9. A product composition obtainable by a process according to claim 7 wherein the product composition comprises at least one glycolipid and wherein the product composition achieves a negative result under the h-CLAT assay.

10. A product composition according to claim 9 comprising a protein concentration of at most 5 mg of protein per g of solids (mg/g) on the basis of the total weight of protein and the total weight of solids in the product composition.

11. A product composition according to claim 9 which has a Gardner colour of at most 5.

12. A personal care formulation comprising a product composition according to claim 9.

13. A home care formulation comprising a product composition according to claim 9.
